(19) United States
(12) Patent Application Publication
(10) Pub. No.: US 2009/0068262 A1
(43) Pub. Date: Mar. 12, 2009

(54) Rapid dissolution of combination products

(76) Inventors: Ilan Zalit, Rosh Haain (IL); Uri Zadok, Herzliyya (IL)

Correspondence Address: KENYON & KENYON LLP, ONE BROADWAY, NEW YORK, NY, 10004, US

(21) Appl. No.: 12/080,761

(22) Filed: Apr. 3, 2008

Related U.S. Application Data
(60) Provisional application No. 60/922,031, filed on Apr. 4, 2007.
(60) Provisional application No. 60/932,613, filed on May 31, 2007.

(51) Int. Cl. A61K 31/404; A61K 9/20; A61P 29/00

(52) U.S. Cl. 424/464; 514/415

(57) ABSTRACT

Provided are rapidly dissolving pharmaceutical oral dosage forms of triptans and NSAIDs, processes for the preparation thereof, and methods of treatment therewith.

### CROSS-REFERENCE TO RELATED APPLICATIONS

This application claims the benefit of U.S. Provisional Application Ser. No. 60/922,031, filed Apr. 4, 2007; U.S. Provisional Application Ser. No. 60/932,613, filed May 31, 2007; and 6_/___,___ [attorney docket no. 01662/A423P3, entitled “Rapid Dissolution of Combination Products”], filed Mar. 21, 2008, the contents of which are hereby incorporated by reference.

### FIELD OF THE INVENTION

The invention encompasses a method for the production of rapid dissolution tablets comprising at least two active pharmaceutical ingredients.

### BACKGROUND OF THE INVENTION

Migraine is the most common cause of vascular headache, and afflicts approximately 15% of women and 6% of men. Typically, migraine is treated with a single drug or a combination of drugs known to be effective in treating migraine. Most drugs effective in the treatment of migraines are members of one of three major pharmacologic classes: anti-inflammatory agents, 5-HT₁ antagonists, and dopamine antagonists. See HARRISON'S PRINCIPLES OF INTERNAL MEDICINE 73-76 (15th ed. 2001).

Sumatriptan succinate has the chemical name 3-[2-(dimethylamino)ethyl]-N-methyl-indole-5-methanesulfonamide succinate and the following chemical structure:

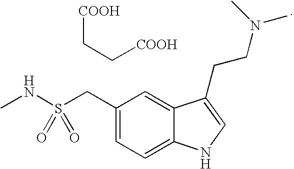

Naproxen sodium has the chemical name (S)-6-methoxy-α-methyl-2-naphthaleneacetic acid, sodium salt, and the following chemical structure:

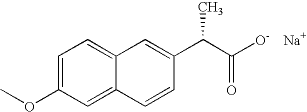

Sumatriptan succinate is a selective 5-hydroxytriptamine₁ receptor subtype agonist currently marketed under the trade name IMITREX® for the acute treatment of migraine attacks with or without aura in adults. Naproxen sodium is a non-steroidal anti-inflammatory drug (NSAID) with analgesic and antipyretic properties. The mechanism of action of the naproxen anion, like that of other NSAIDs, is not completely understood but may be related to prostaglandin synthetase inhibition. Both actives have been combined in a single tablet, which is expected to be marketed under the trade name TREXIMA®. Current treatments for migraines include a range of over-the-counter analgesics and triptans. Recent reports have indicated that combination therapies of triptans and NSAIDs greatly improve the relief of migraines in patients. See, e.g., U.S. Pat. Nos. 5,872,145; 6,060,499; 6,586,458.

U.S. Publication No. 2004/0180089 (“the '089 publication”) refers to a dosage form containing a triptan and an NSAID as therapeutic agents, where the triptan and the NSAID are segregated into separate layers, in a side-by-side arrangement, such that the dissolution of each therapeutic agent occurs independently of the other. The '089 publication, p. 1, å 6-7. The '089 publication suggests that in the stomach, the NSAID naproxen may form a gel-like matrix, which retards the dissolution of the triptan unless the two therapeutic agents are maintained in distinct side-by-side layers. Id. The '089 publication addresses this dissolution problem by separating the triptan and the NSAID into separate distinct layers, such that each layer contains at least 90% of either the triptan or the NSAID, as opposed to a single layer tablet matrix containing both agents or one layer forming a core surrounded by the other layer. The '089 publication, p. 1, å 10. The '089 publication discloses that such an arrangement provides for the independent dissolution of each therapeutic agent, such that the dissolution rates approximate the rates if the drugs were given separately. Id.

However, this technique of preparing bilayer tablets requires specialized tabletting equipment, such as a rotary bilayer tablet press. Furthermore, the process itself is cumbersome and requires several stages. For example, the NSAID layer is loaded first, compacted using a minimal compaction force, and the triptan layer is added next, and the tablet compacted using a final compaction force such that a tablet with the desired mechanical properties is achieved. See, e.g., the '089 publication, p. 3, å 33.

U.S. Publication No. 2007/0184109 (“the '109 publication”) also refers to pharmaceutical compositions comprising a 5HT₁ receptor agonist (such as sumatriptan succinate) or a pharmaceutically acceptable derivative thereof in combination with an NSAID (such as naproxen sodium) or pharmaceutically acceptable derivative thereof. The '109 publication compares the dissolution of sumatriptan succinate in two compositions of sumatriptan succinate and naproxen sodium, the first having the sumatriptan succinate and the naproxen sodium in the same zone and the second having the sumatriptan succinate and the naproxen sodium in separate layers of a bilayer tablet. '109 publication, pp. 9-10, tables 3 and 4. The '109 publication states that the dissolution of the first composition was “slower than expected,” and that the slow dissolution “led to the conclusion that the naproxen sodium was disabling the dissolution of the sumatriptan granules when blended together.” See id. at p. 9, å 97. As to the second composition, the '109 publication states that “[c]ombining the two formulations as separate layers in a bilayer tablet allowed for the rapid release of sumatriptan succinate from the tablet, without as much interference from naproxen sodium.” See id. at p. 10, å 100.

Therefore, there is a need for an efficient simple process which does not require specialized equipment, while maintaining the dissolution properties of each of the therapeutic agents.

### SUMMARY OF THE INVENTION

In one embodiment, the invention encompasses an oral dosage form comprising a therapeutically effective amount of a triptan and a therapeutically effective amount of an NSAID, wherein the dosage form includes at least one zone in which particles of the triptan and the NSAID are admixed, and not less than about 60% of the triptan dissolves in 30 minutes in 200 ml of 0.01N HCl solution in a USP Type II Dissolution Apparatus at a rotation speed of 40 rpm and a temperature of 37° C. Preferably, not less than about 70% of the triptan dissolves in 30 minutes, and more preferably not less than about 80% of the triptan dissolves in 30 minutes.

In one preferred embodiment, the oral dosage form further comprises at least one additive selected from the group consisting of (i) a carbonate, (ii) a bicarbonate, and (iii) a superdisintegrant, with the proviso that the superdisintegrant is not croscarmellose sodium.

In another preferred embodiment, the oral dosage form is in the form of a porous tablet. Preferably, the porosity of the tablet is at least about 10%.

In yet another embodiment, the invention encompasses an oral dosage form comprising: (a) a therapeutically effective amount of a triptan, (b) a therapeutically effective amount of an NSAID, and (c) at least one additive selected from the group consisting of (i) a carbonate, (ii) a bicarbonate, and (iii) a superdisintegrant, wherein the dosage form includes at least one zone in which particles of the triptan and the NSAID are admixed, the additive is present in an amount of more than 10% by weight of the oral dosage form, and the superdisintegrant is not croscarmellose sodium.

In another embodiment, the invention encompasses an oral dosage form comprising: (a) a therapeutically effective amount of a triptan, (b) a therapeutically effective amount of an NSAID, and (c) at least one additive selected from the group consisting of (i) a carbonate, (ii) a bicarbonate, and (iii) a superdisintegrant, wherein the dosage form includes at least one zone in which particles of the triptan and the NSAID are admixed, the ratio of the amount of NSAID to the total amount of additives is less than about 5, and the superdisintegrant is not croscarmellose sodium.

In another embodiment, the invention encompasses a method for preparing the above-described oral dosage forms comprising separately granulating each of the triptan and the NSAID, combining the separate granulates with at least one extragranular pharmaceutically acceptable excipient to form a mixture, and compacting the mixture into the oral dosage form.

In another embodiment, the invention encompasses a method for preparing the above-described oral dosage forms comprising granulating one of the triptan or the NSAID with at least one pharmaceutically acceptable excipient to form a granulate, adding to the granulate a dry blend comprising at least the other of the triptan or the NSAID and at least one pharmaceutically acceptable excipient to form a mixture, and compacting the mixture into the oral dosage form.

In another embodiment, the invention encompasses a method for preparing the above-described oral dosage forms comprising coating an inert core with a layer comprising a triptan and an NSAID.

In another embodiment, the invention encompasses a method of treating acute migraines comprising administering a therapeutically effective amount of at least one of the above-described dosage forms to a patient in need thereof. Preferably, the triptan is sumatriptan and the NSAID is naproxen.

### BRIEF DESCRIPTION OF THE DRAWINGS

FIG. 1 illustrates exemplary tablet forms of triptan and NSAID. In FIG. 1, “A” comprises the triptan and, optionally, at least one pharmaceutically acceptable excipient; “B” comprises the NSAID and, optionally, at least one pharmaceutically acceptable excipient; and “C” comprises an additional active material and/or at least one pharmaceutically acceptable excipient.

FIG. 2 illustrates comparative dissolution of 2 separate tablets of sumatriptan and naproxen (Compositions 10 and 11), homogeneous compositions of sumatriptan and naproxen with sodium bicarbonate and the superdisintegrant crospovidone (Compositions 6 and 8), and a bi-layer tablet of sumatriptan and naproxen (Composition 13).

### DETAILED DESCRIPTION OF THE INVENTION

The invention addresses the above-described shortcomings of the prior art by providing rapidly dissolving tablets comprising a triptan and an NSAID, wherein the triptan and the NSAID are present in a single zone and each of the triptan and the NSAID maintains its individual dissolution characteristics irrespective of the presence of the other.

When naproxen or a similar NSAID and a triptan are in a non-segregated tablet, it has been reported that the NSAID, which is usually present in a high dose compared with the triptan, tends to form a gel-like matrix in the presence of an aqueous medium. The gel-like matrix erodes slowly and is likely to entrap the triptan, thereby delaying its release. It has now been surprisingly found, however, that when rapidly dissolving tablets are used, the formation of the gel is prevented, allowing for the rapid dissolution of the triptan.

As used herein, unless otherwise defined, the term “triptan” means a triptan or a pharmaceutically acceptable salt or solvate thereof.

As used herein, unless otherwise defined, the term “NSAID” means an NSAID or a pharmaceutically acceptable salt or solvate thereof.

As used herein, unless otherwise defined, the term “sumatriptan” means sumatriptan free base or a pharmaceutically acceptable salt or solvate thereof. A preferred form of sumatriptan is sumatriptan succinate.

As used herein, unless otherwise defined, the term “naproxen” means naproxen free acid or a pharmaceutically acceptable salt or solvate thereof. A preferred form of naproxen is naproxen sodium.

As used herein, unless otherwise defined, the term “therapeutically effective dosage” means a dosage sufficient to treat migraines. The person of ordinary skill in the art would be able to determine a therapeutically effective dosage based upon the description herein coupled with the general knowledge in the art. For a particular patient, a therapeutically effective dosage may depend on variables such as age, gender, weight, and extent of condition.

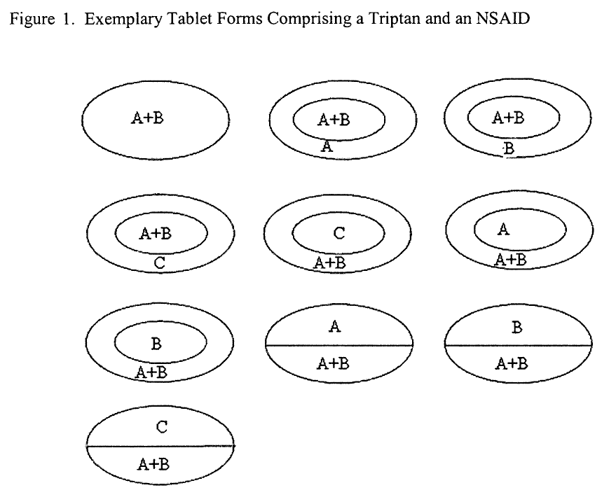

As used herein, unless otherwise defined, the term “zone” means a region in a dosage form that has a distinct composition. Preferably, the region has a substantially homogeneous composition, i.e., the region contains a substantially homogeneous mixture of particles of triptan and particles of NSAID. In one preferred embodiment, the region contains a substantially homogeneous mixture of granulates of triptan, granulates of NSAID, and extragranular excipients. For example, a zone may be a core, a coating layer, or other type of layer in the oral dosage form, examples of which are depicted in FIG. 1.

The invention encompasses oral dosage forms and compositions comprising a triptan and an NSAID. Suitable triptans include, but are not limited to, sumatriptan, eletriptan, rizatriptan, frovatriptan, almotriptan, zolmitriptan, and naratriptan. Preferably, the triptan is sumatriptan. Suitable NSAIDs include, but are not limited to, flurbiprofen, ketoprofen, naproxen, oxaprozin, etodolac, indomethacin, ketorolac, nabumetone, mefanamic acid, piroxicam, lornoxicam, meloxicam, rofecoxib, celecoxib, etoricoxib, and valdecoxib. Preferably, the NSAID is naproxen or rofecoxib. More preferably, the NSAID is naproxen.

Typically, the oral dosage forms of the invention are in the form of rapidly dissolving tablets in which the triptan and the NSAID are combined in a single zone, while each of the triptan and the NSAID maintains its individual dissolution characteristics irrespective of the presence of the other.

Typically, a rapid dissolution will be understood to mean that not less than about 60% of the triptan in the oral dosage form dissolves in acidic media in 30 minutes. Preferably not less than about 70% of the triptan dissolves in acidic media in 30 minutes, and more preferably not less than about 80% of the triptan dissolves in acidic media in 30 minutes. As used herein, unless otherwise defined, the term “acidic media” means 200 ml of 0.01N HCl solution in a USP Type II Dissolution Apparatus at a rotation speed of 40 rpm.

In one embodiment of the invention, the rapid dissolution of the oral dosage form is achieved through the use of an effervescent couple. The effervescent couple comprises a basic ingredient and an acidic ingredient, the basic ingredient liberating carbon dioxide when it and the acidic ingredient are contacted with water. The effervescent couple can be formed, for example, by including both the base component and the acid component as extra-granular excipients in the oral dosage form. In another example, the effervescent couple can be formed by including only the base component as an extra-granular excipient in the oral dosage form, and using stomach acid as the acid component, i.e., the base component reacts with stomach acid to form the effervescent couple once the oral dosage form is administered to a patient.

Typically, the base component is a carbonate or a bicarbonate. Suitable bicarbonates include, but are not limited to, ammonium bicarbonate, and alkali metal bicarbonates such as potassium bicarbonate and sodium bicarbonate. Preferably, the bicarbonate is sodium bicarbonate. Typically, the bicarbonate is present in an amount of more than 10% to about 20% by weight of the zone in which both particles of triptan and particles of NSAID are present. Preferably, the bicarbonate is present in an amount of about 11% to about 17% by weight of the zone in which both particles of triptan and particles of NSAID are present.

The acid component, when included in the oral dosage form, may be any pharmaceutically acceptable acid. Suitable acids include, but are not limited to, ascorbic acid, citric acid, tartaric acid, succinic acid, fumaric acid, malic acid, lactic acid, propionic acid, sorbic acid, and benzoic acid. Preferably, the acid is ascorbic acid, citric acid or tartaric acid. Typically, the acid component is present in an amount so as to produce an approximately a 1:1 molar ratio between the basic groups on the base component and the acidic groups on the acid component.

In another embodiment of the invention, the rapid dissolution of the oral dosage form may be achieved by using a large amount of superdisintegrant as an extragranular excipient, with the proviso that the superdisintegrant is not croscarmellose sodium. Suitable superdisintegrants include, but are not limited to, crospovidone and sodium starch glycolate. Preferably, the superdisintegrant is crospovidone. Typically, the crospovidone is present in an amount of about 7% to about 20% by weight of the zone in which both particles of triptan and particles of NSAID are present, and preferably more than 10% to about 17% by weight of the zone in which both particles of triptan and particles of NSAID are present. The skilled artisan would appreciate that the appropriate amount of superdisintegrant varies with the type of superdisintegrant used, as well as the properties of the active materials and other excipients used, and can determine the appropriate amount of superdisintegrant based upon these factors.

Optionally, the rapid dissolution of the oral dosage form may be achieved by using a mixture of additives as extra-granular excipients, i.e., a mixture of carbonate, bicarbonate, and/or superdisintegrant. Typically, the additives are present in a total amount of more than 10% by weight of the zone in which both particles of triptan and particles of NSAID are present. Preferably, the additives are present in a total amount of more than 10% to about 60% by weight, more preferably more than 10% to about 50% by weight, and most preferably more than 10% to about 40% by weight of the zone in which both particles of triptan and particles of NSAID are present.

Typically, the ratio of the amount of NSAID to the total amount of additives is less than about 5. Preferably, the ratio of the amount of NSAID to the total amount of additives is about 1 to about 4.5, more preferably about 2 to about 4.5, and most preferably about 2.5 to about 4.5.

In another embodiment of the invention, the rapid dissolution of the oral dosage form may be achieved by producing a highly porous tablet. An increased dissolution rate can be obtained by increasing the porosity (void spaces) of the tablet. Void spaces in the tablet matrix facilitate the permeation of water to rapidly erode the entire tablet. This may be achieved by adding a volatilizable adjuvant to the composition, which is subsequently removed by sublimation or thermal decomposition. See, e.g., U.S. Pat. Nos. 3,885,026, 5,529,789, 5,853,758; Aly, A. M., Semreen M., and Qato, M. K., “Superdisintegrants for solid dispersion to produce rapidly disintegrating tenoxicam tablets via camphor sublimation,” Pharmaceutical Technology, January 2005, pp. 68-78; Koizumi K. et al., “New method of preparing high-porosity rapidly saliva soluble compressed tablets using mannitol with camphor, a subliming material,” Int. J. Pharm. 152 (1997) pp. 127-131; Gohel, M. et al., “Formulation design and optimization of mouth dissolve tablets of nimesulfide using vacuum drying technique,” AAAPS PharmSciTech 2004, 5(3) Article 36, all of which are hereby incorporated by reference. The degree of porosity may be expressed as the percent of weight lost from the dosage form following removal of the volatilizable adjuvant.

Preferably, the porosity of the tablet is at least about 10%, i.e., upon removal of the volatilizable adjuvant, 10% of the tablet weight is lost. The weight loss (and thus the porosity of the tablet) can be measured, for example, by techniques disclosed in the articles mentioned in the immediately preceding paragraph. The desired weight loss may be achieved by incorporating into the extra granular excipients a solid adjuvant which is volatilizable at either elevated temperatures, or reduced pressures, or a combination of both. Suitable volatilizable adjuvants include materials that undergo sublimation and materials that thermally decompose to gases. Materials that undergo sublimation include, but are not limited to, camphor, menthol, benzoic acid, urethane, urea, vanillin, tetramethylene tetramine, and naphthalene. Preferably, the material that undergoes sublimation is camphor or menthol. Materials that thermally decompose to gases include, for example, ammonium bicarbonate.

In another embodiment of the invention, the rate of rapid dissolution may be adjusted by controlling the hardness of the tablet. Preferably, the hardness of the tablet is about 10 to about 30 SCU, and more preferably about 12 to about 25 SCU. Hardiness is typically measured by the method disclosed in REMINGTON—THE SCIENCE AND PRACTICE OF PHARMACY, 882-883 (20th ed. 2000), hereby incorporated by reference.

The compositions described herein may be prepared in accordance with methods that are standard in the art. Preferably, the triptan and NSAID are granulated separately using any acceptable granulation method, using, for example, a high-shear granulator. Any acceptable granulation solvent may be employed, such as, but not limited to, water, ethanol, isopropanol, or any combination thereof. More preferably, the granulation solvent is water.

Optionally, the NSAID granulate may be mixed with colloidal silicon dioxide before adding the triptan granulate and the remaining inactive ingredients.

Apart from the active ingredients, any acceptable pharmaceutical excipient may be used. For example, the composition may comprise binders (such as polyvinylpyrrolidone), disintegrants (such as crospovidone, microcrystalline cellulose, and sodium starch glycolate), lubricants (such as magnesium stearate and hydrogenated vegetable oil), and fillers (such as lactose and mannitol). The tablets may optionally be coated.

The invention further encompasses a method of treatment comprising administering the pharmaceutical formulation to a mammal. Preferably, the mammal is a human. Preferably, the pharmaceutical formulation comprises sumatriptan succinate and naproxen sodium in the form of a rapidly dissolving tablet in which the sumatriptan succinate and the naproxen sodium are combined in a single zone and is administered to treat acute migraines, which is expected to be the approved use for TREXIMA®.

The amount of active compounds administered and the dosing regimen used will depend on the particular drug selected, the age and general condition of the subject being treated, the severity of the subject's condition, and the judgment of the prescribing physician.

Having described the invention with reference to certain preferred embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

In the following examples several fixed-dose combinations of sumatriptan succinate and naproxen sodium have been prepared.

### Example 1

### Granulation of Sumatriptan Succinate

The composition for sumatriptan succinate granulation is provided in Table 1.

TABLE 1
Composition of sumatriptan succinate granulation.

| | Weight % | Amount | Total |
|---|---|---|---|
| Ingredient | (w/w) | (mg/tablet) | weight (g) |
| Sumatriptan succinate | 56.1 | 119.00 | 29.75 |
| Lactose monohydrate | 24.1 | 51.00 | 12.75 |
| Microcrystalline cellulose | 19.8 | 42.00 | 10.5 |
| Water | — | QS | |
| Total | 100.0 | 212.00 | 53.0 |

Sumatriptan succinate, lactose monohydrate and microcrystalline cellulose were mixed for 2 minutes in a high shear granulator at a mixer speed of 460 rpm. Water was added until a suitable granulation was achieved at a mixer speed of 920 rpm and a chopper speed of 2220 rpm. The resulting wet granulate was transferred to a fluid bed drier (Glatt-2), with an inlet temperature of 60° C. and an outlet temperature of 35° C., and dried. Loss on drying (LOD) was determined using a Mettler-Toledo HR73 Halogen Moisture Analyzer, and was found to be 0.69%. The dried granulate was then milled using an 18 mesh screen.

### Example 2

### Granulation of Naproxen Sodium 500 mg

The composition for naproxen sodium granulation is provided in Table 2.

TABLE 2
Composition of naproxen sodium granulation.

| | Weight % | Amount | Total |
|---|---|---|---|
| Ingredient | (w/w) | (mg/tablet) | weight (g) |
| Naproxen sodium | 87.0 | 500.00 | 125.0 |
| Microcrystalline cellulose | 8.7 | 50.00 | 12.5 |
| Povidone | 4.3 | 25 | 6.25 |
| Water | — | QS | |
| Total | 100.0 | 575.00 | |

Naproxen sodium, microcrystalline cellulose and povidone were mixed for 2 minutes in a high shear granulator at a mixer speed of 460 rpm. Water was added until a suitable granulation was achieved at a mixer speed of 920 rpm and a chopper speed of 2220 rpm. The resulting wet granulate was transferred to a fluid bed drier (Glatt-2), with an inlet temperature of 60° C. and an outlet temperature of 35° C., and dried. Loss on drying (LOD) was determined using a Mettler-Toledo HR73 Halogen Moisture Analyzer, and was found to be 2.84%. The dried granulate was then milled using an 18 mesh screen.

### Example 3

### Preparation of Compositions Containing Granulates of Naproxen and Sumatriptan

The granulates from Examples 1-2 were combined in pharmaceutical compositions according to Tables 3a, 3b, 3c, and 3d. About 15-20 tablets were prepared from each batch.

TABLE 3a
Comparative formulations of naproxen and sumatriptan without
sodium bicarbonate or the superdisintegrant crospovidone.

| | | Composi- | Composi- | Composi- |
|---|---|---|---|---|
| | Ingredient | tion 1 | tion 2 | tion 12 |
| Part I | Naproxen granulate | 68.05% | 64.25% | 57.10% |
| Part II | Sumatriptan | 25.08% | 23.695 | 21.05% |
| | granulate | | | |
| Part III | Microcrystalline | 2.37% | 2.23% | 1.99% |
| | cellulose | | | |
| | Lactose anhydrous | 2.37% | 2.23% | 1.99% |
| | Croscarmellose | 1.18% | 5.59% | 13.90% |
| | sodium | | | |
| | Tartaric acid | — | — | — |
| | Sodium bicarbonate | — | — | — |
| | Crospovidone | — | — | — |
| | Colloidal silicon | — | — | 1.98% |
| | dioxide | | | |
| | [AEROSIL ® 200] | | | |
| | Menthol | — | — | — |
| Part IV | Magnesium stearate | 0.95% | 2.01% | 1.99% |
| Total | | 100.00% | 100.00% | 100.00% |

TABLE 3b
Formulations of naproxen and sumatriptan with sodium bicarbonate
or the effervescent couple sodium bicarbonate and tartaric acid.

| | | Composi- | Composi- |
|---|---|---|---|
| | Ingredient | tion 3 | tion 4 |
| Part I | Naproxen granulate | 52.80% | 57.21% |
| Part II | Sumatriptan granulate | 19.47% | 21.09% |
| Part III | Microcrystalline cellulose | 3.67% | 3.98% |
| | Lactose anhydrous | 3.67% | 3.98% |
| | Croscarmellose sodium | — | — |
| | Tartaric acid | 8.82% | — |
| | Sodium bicarbonate | 9.92% | 11.94% |
| | Crospovidone | — | — |
| | Colloidal silicon dioxide | — | — |
| | [AEROSIL ® 200] | | |
| | Menthol | — | — |
| Part IV | Magnesium stearate | 1.65% | 1.79% |
| Total | | 100.00% | 100.00% |

TABLE 3c
Formulations of naproxen and sumatriptan
with the superdisintegrant crospovidone.

| | | Composi- | Composi- |
|---|---|---|---|
| | Ingredient | tion 5 | tion 7 |
| Part I | Naproxen granulate | 63.54% | 57.10% |
| Part II | Sumatriptan granulate | 23.43% | 21.05% |
| Part III | Microcrystalline cellulose | 2.21% | 1.99% |
| | Lactose anhydrous | 2.21% | 1.99% |
| | Croscarmellose sodium | — | — |
| | Tartaric acid | — | — |
| | Sodium bicarbonate | — | — |
| | Crospovidone | 2.21% | 13.90% |
| | Colloidal silicon dioxide | — | 1.99% |
| | [AEROSIL ® 200] | | |
| | Menthol | 4.42% | — |
| Part IV | Magnesium stearate | 2.03% | 1.99% |
| Total | | 100.00% | 100.00% |

The porosity of composition 5 was measured to be about 4.4%.

TABLE 3d
Formulations of naproxen and sumatriptan with both sodium
bicarbonate and the superdisintegrant crospovidone.

| | | Composi- | Composi- | Composi- |
|---|---|---|---|---|
| | Ingredient | tion 6 | tion 8 | tion 9* |
| Part I | Naproxen granulate | 58.26% | 57.10% | 57.10% |
| Part II | Sumatriptan | 21.48% | 21.05% | 21.05% |
| | granulate | | | |
| Part III | Microcrystalline | 2.03% | 1.99% | 1.99% |
| | cellulose | | | |
| | Lactose anhydrous | 2.03% | 1.99% | 1.99% |
| | Croscarmellose | — | — | — |
| | sodium | | | |
| | Tartaric acid | — | — | — |
| | Sodium bicarbonate | 12.16% | 11.92% | 11.92% |
| | Crospovidone | 2.03% | 1.98% | 1.98% |
| | Colloidal silicon | — | 1.98% | 1.98% |
| | dioxide | | | |
| | [AEROSIL ® 200] | | | |
| | Menthol | — | — | — |
| Part IV | Magnesium stearate | | 1.99% | 1.99% |
| Total | | 100.00% | 100.00% | 100.00% |

*In this composition the colloidal silicon dioxide used was AEROSIL ® R 972. All other compositions in Tables 3a to 3d contained AEROSIL ® 200.

Part III ingredients were combined and mixed. Naproxen granulate was added and mixed for 5 minutes. Sumatriptan granulate was added and mixed for 5 minutes. Magnesium stearate was added and mixed for 2 minutes. Compositions were tabletted using a Kilian SP300 tabletting machine, with a mean hardness of about 20-24 SCU (high hardness, HH). Compositions 8 and 9 were tabletted to a mean hardness of about 13-15 SCU (low hardness, LH).

Tablets from composition 5 were then placed in a vacuum oven heated to about 35° C. for about 5 hours.

### Example 4

### Preparation of a Comparative Composition Containing Sumatriptan

The granulation of Example 1 was used in a composition according to Table 4. About 15 tablets were prepared.

TABLE 4
Composition containing sumatriptan succinate

| | Ingredient | Composition 10 |
|---|---|---|
| Part I | Sumatriptan granulate | 80.00% |
| Part II | Microcrystalline cellulose | 7.55% |
| | Lactose anhydrous | 7.55% |
| | Croscarmellose sodium | 3.77% |
| Part III | Magnesium stearate | 1.13% |
| Total | | 100.00% |

Ingredients of Part II were combined and mixed for 5 minutes. The resulting sumatriptan granulation was then added and blended for 5 minutes. Magnesium stearate was finally added to the sumatriptan granulation, and blended for 2 minutes. Tablets were prepared using a Kilian SP300 tabletting machine, to a mean hardness of about 20 SCU.

### Example 5

### Preparation of a Comparative Composition Containing Naproxen

The granulation of Example 2 was used in a composition according to Table 5. About 15 tablets were prepared.

TABLE 5
Composition containing naproxen

| | Ingredient | Composition 11 |
|---|---|---|
| Part I | Naproxen granulate | 91.13% |
| Part II | Microcrystalline cellulose | 3.17% |
| | Lactose anhydrous | 3.17% |
| | Croscarmellose sodium | 1.58% |
| Part III | Magnesium stearate | 0.95% |
| Total | | 100.00% |

Ingredients of Part II were combined and mixed for 5 minutes. Naproxen granulation was then added and blended for 5 minutes. Magnesium stearate was finally added to the naproxen granulation, and blended for 2 minutes. Tablets were prepared using a Killian SP300 tabletting machine, to a mean hardness of about 20 scu.

### Example 6

### Preparation of a Bi-Layer Tablet Containing Sumatriptan and Naproxen

The granulations from examples 1-2 were used in a composition according to Table 6. About 6 tablets were prepared.

TABLE 6
Bi-layer tablet containing sumatriptan and naproxen

| | Ingredient | Composition 13 |
|---|---|---|
| Part I | Naproxen granulate | 67.97% |
| Part II | Sumatriptan granulate | 25.06% |
| Part III | Microcrystalline cellulose | 2.36% |
| | Lactose anhydrous | 2.36% |
| | Croscarmellose sodium | 1.18% |
| Part IIV | Magnesium stearate | 1.07% |
| Total | | 100.00% |

Ingredients of Part III were combined and mixed. ⅔ of the total weight were mixed for 5 minutes with granulate of Part I, 6 mg of magnesium stearate were added, and the combined mixture mixed for 2 minutes. The resulting first admixture was poured into the matrix of a manual press, and lightly pressed. The remainder of the Part III mix were mixed for 5 minutes with the granulate of Part II, and then 3 mg of magnesium stearate were added, and the admixture was mixed for 2 minutes. The resulting second admixture was poured on top of the first admixture inside the matrix. The total composition was pressed using a manual press. The hardness of the resulting tablets was not determined.

### Example 7

### Dissolution of Tablets

Dissolution of the sumatriptan in tablets prepared in Example 3 was studied using a USP Apparatus 2 (paddles), rotation speed of 40 rpm, at a temperature of 37° C. See U.S. PHARMACOPEIA, 2413-2414 (28th ed. 2005), hereby incorporated by reference. The medium used was 0.01 N HCl or a buffered solution at pH 6 with 0.15% sodium dodecyl sulfate. In some cases, 0.01N HCl was used for 30 minutes, neutralized with a sodium phosphate and sodium hydroxide solution, buffered to pH 6 and 0.15% sodium dodecyl sulfate added. The dissolution profile of the sumatriptan in these compositions was compared with the dissolution of sumatriptan in a tablet of sumatriptan prepared according to Example 4 in the presence of a separate tablet of naproxen prepared according to Example 5, as well as the dissolution of sumatriptan in a bilayer tablet of sumatriptan and naproxen prepared according to Example 6. The results are summarized in Table 7 below.

TABLE 7
Dissolution of sumatriptan following
exposure to 0.01N HCl for 30 minutes

| | Formulation | | |
|---|---|---|---|
| | (total % carbonate, | | |
| | bicarbonate and | Hardness | % |
| | superdisintegrant) | (SCU) | Dissolved |
| Sumatriptan and naproxen | Composition 1 | 22-25 | 10** |
| without a carbonate, | Composition 2 | 22-25 | 24 |
| bicarbonate or the | Composition 12 | 15-17 | 16 |
| superdisintegrant | | | |
| crospovidone | | | |
| Sumatriptan and naproxen | Composition 3 | 22-25 | 48 |
| with sodium bicarbonate or | (9.92%) | | |
| the effervescent couple | Composition 4 | 22-25 | 77 |
| sodium bicarbonate and | (11.94%) | | |
| tartaric acid | | | |
| Sumatriptan and naproxen | Composition 5 | 22-25 | 13 |
| with the superdisintegrant | (2.21%) | | |
| crospovidone | Composition 7 | 13-17 | 75 |
| | (13.90%) | | |
| Sumatriptan and naproxen | Composition 6 | 13-17 | 85** |
| with sodium bicarbonate | (14.19%) | | |
| and the superdisintegrant | Composition 8 LH | 13-17 | 95* |
| | (13.90%) | | |
| crospovidone | Composition 8 HH | 22-25 | 89** |
| | (13.90%) | | |
| | Composition 9 LH | 13-15 | 102 |
| | (13.90%) | | |
| | Composition 9 HH | 22-25 | 88 |
| | (13.90%) | | |
| Sumatriptan and naproxen | Composition 10 (in | — | 89* |
| in separate tablets | the presence of | | |
| Bi-layer tablet of | composition 11) | | |
| sumatriptan and naproxen | Composition 13 | — | 68 |

*average of 3 experiments;
**average of 2 experiments;
LH = low hardness; HH = high hardness

As illustrated in Table 7 above, compositions having less than 10% of carbonate, bicarbonate, or superdisintegrant release about 25% or less of the sumatriptan following exposure to 0.01N HCL for 30 minutes. Compositions having more than 10% of carbonate, bicarbonate, or superdisintegrant, however, release about 50% to 100% of the sumatriptan following exposure to 0.01N HCl for 30 minutes. The dissolution profile of sumatriptan in the compositions having more than 10% of carbonate, bicarbonate, or superdisintegrant is comparable to the dissolution profile of sumatriptan when it is administered separately from naproxen, either in a separate tablet or in a bi-layer tablet wherein the naproxen and the sumatriptan are in separate layers.

### Example 8

### Dissolution of Tablets

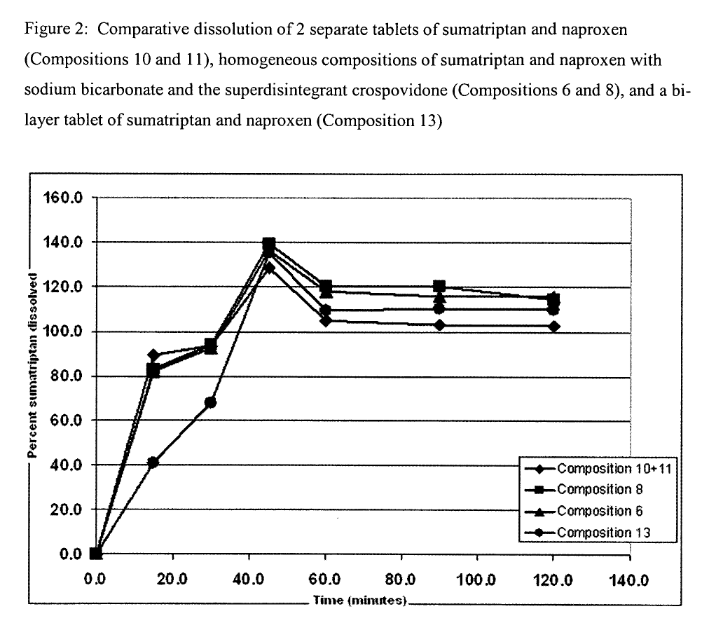

Comparative dissolution of 2 separate tablets of sumatriptan and naproxen (Compositions 10 and 11), homogeneous compositions of sumatriptan and naproxen with sodium bicarbonate and the superdisintegrant crospovidone (Compositions 6 and 8), and a bi-layer tablet of sumatriptan and naproxen (Composition 13) was carried out using a USP Apparatus II (paddles), rotation speed 40 r.p.m, at a temperature of 37° C. Tablets were initially placed in 200 mL 0.01N HCl solution for 30 minutes. The solution was then neutralized using 10 mL of phosphate neutralizing buffer (60 g/L sodium dihydrogen phosphate, titrated to pH 6 with sodium hydroxide, and sodium hydroxide added until a pH of 10 is reached). This was followed by addition of 700 mL phosphate buffer (6 g/L sodium dihydrogen phosphate, pH adjusted to 6 with sodium hydroxide, and 0.15% (w/v) sodium lauryl sulfate added). Samples were drawn 15, 30, 45, 60, 90, and 120 minutes after the experiment was started. Results are shown in FIG. 2.

We claim:

1. An oral dosage form comprising a therapeutically effective amount of a triptan and a therapeutically effective amount of an NSAID, wherein the dosage form includes at least one zone in which particles of the triptan and the NSAID are admixed, and not less than about 60% of the triptan dissolves in 30 minutes in 200 ml of 0.01N HCl solution in a USP Type II Dissolution Apparatus at a rotation speed of 40 rpm and a temperature of 37° C.

2. The oral dosage form of claim 1, wherein the triptan is sumatriptan, eletriptan, rizatriptan, frovatriptan, almotriptan, zolmitriptan, or naratriptan.

3. The oral dosage form of claim 1, wherein the triptan is sumatriptan.

4. The oral dosage form of claim 1, wherein the NSAID is flurbiprofen, ketoprofen, naproxen, oxaprozin, etodolac, indomethacin, ketorolac, nabumetone, mefanamic acid, piroxicam, lornoxicam, meloxicam, rofecoxib, celecoxib, etoricoxib, or valdecoxib.

5. The oral dosage form of claim 1, wherein the NSAID is naproxen or rofecoxib.

6. The oral dosage form of claim 1, wherein the triptan is sumatriptan and the NSAID is naproxen.

7. The oral dosage form of claim 1, wherein not less than about 70% of the triptan dissolves in 30 minutes.

8. The oral dosage form of claim 1, wherein not less than about 80% of the triptan dissolves in 30 minutes.

9. The oral dosage form of claim 1, further comprising at least one additive selected from the group consisting of (i) a carbonate, (ii) a bicarbonate, and (iii) a superdisintegrant, with the proviso that the superdisintegrant is not croscarmellose sodium.

10. The oral dosage form of claim 9, wherein the bicarbonate is ammonium bicarbonate, potassium bicarbonate, or sodium bicarbonate.

11. The oral dosage form of claim 9, wherein the bicarbonate is sodium bicarbonate.

12. The oral dosage form of claim 9, wherein the bicarbonate or carbonate is present in an amount of more than 10% to about 20% by weight of the zone in which particles of the triptan and the NSAID are admixed.

13. The oral dosage form of claim 12, wherein the bicarbonate or carbonate is present in an amount of about 11% to about 17% by weight of the zone in which particles of the triptan and the NSAID are admixed.

14. The oral dosage form of claim 9, wherein the additive is a bicarbonate.

15. The oral dosage form of claim 14, further comprising a pharmaceutically acceptable acid.

16. The oral dosage form of claim 15, wherein the pharmaceutically acceptable acid is ascorbic acid, citric acid, tartaric acid, succinic acid, fumaric acid, malic acid, lactic acid, propionic acid, sorbic acid, or benzoic acid.

17. The oral dosage form of claim 9, wherein the superdisintegrant is crospovidone or sodium starch glycolate.

18. The oral dosage form of claim 9, wherein the superdisintegrant is present in an amount of about 7% to about 20% by weight of the zone in which particles of the triptan and the NSAID are admixed.

19. The oral dosage form of claim 18, wherein the superdisintegrant is present in an amount of more than 10% to about 17% by weight of the zone in which particles of the triptan and the NSAID are admixed.

20. The oral dosage form of claim 9, wherein the additive is present in an amount of more than 10% by weight of the zone in which particles of the triptan and the NSAID are admixed.

21. The oral dosage form of claim 9, wherein the triptan is in granulate form and the additive is extra-granular.

22. The oral dosage form of claim 21, wherein the NSAID is in granulate form.

23. The oral dosage form of claim 1 in the form of a porous tablet.

24. The oral dosage form of claim 23, wherein the porosity of the tablet is at least about 10%.

25. The oral dosage form of claim 23, further comprising a volatilizable adjuvant.

26. The oral dosage form of claim 25, wherein the volatilizable adjuvant is camphor, menthol, benzoic acid, urethane, urea, vanillin, tetramethylene tetramine, naphthalene, or ammonium bicarbonate.

27. A method for preparing the oral dosage form of claim 1 comprising separately granulating each of the triptan and the NSAID, combining the separate granulates with at least one extragranular pharmaceutically acceptable excipient to form a mixture, and compacting the mixture into the oral dosage form.

28. A method for preparing the oral dosage form of claim 1 comprising granulating one of the triptan or the NSAID with at least one pharmaceutically acceptable excipient to form a granulate, adding to the granulate a dry blend comprising at least the other of the triptan or the NSAID and at least one pharmaceutically acceptable excipient to form a mixture, and compacting the mixture into the oral dosage form.

29. A method for preparing the oral dosage form of claim 1 comprising coating an inert core with a layer comprising a triptan and an NSAID.

30. A method of treating acute migraines comprising administering a therapeutically effective amount of the oral dosage form of claim 1 to a patient in need thereof.

31. An oral dosage form comprising:
(a) a therapeutically effective amount of a triptan,
(b) a therapeutically effective amount of an NSAID, and
(c) at least one additive selected from the group consisting of (i) a carbonate, (ii) a bicarbonate, and (iii) a superdisintegrant, wherein
the dosage form includes at least one zone in which particles of the triptan and the NSAID are admixed,
the additive is present in an amount of more than 10% by weight of the zone in which particles of the triptan and the NSAID are admixed, and
the superdisintegrant is not croscarmellose sodium.

32. The oral dosage form of claim 31, wherein the superdisintegrant is crospovidone.

33. An oral dosage form comprising:
(a) a therapeutically effective amount of a triptan,
(b) a therapeutically effective amount of an NSAID, and
(c) at least one additive selected from the group consisting of (i) a carbonate, (ii) a bicarbonate, and (iii) a superdisintegrant, wherein
the dosage form includes at least one zone in which particles of the triptan and the NSAID are admixed,
the ratio of the amount of NSAID to the amount of additive is less than about 5, and
the superdisintegrant is not croscarmellose sodium.

34. The oral dosage form of claim 33, wherein the superdisintegrant is crospovidone.
